(19) United States
(12) Patent Application Publication
(10) Pub. No.: US 2011/0282094 A1
(43) Pub. Date: Nov. 17, 2011

(54) PROCESS FOR PREPARATION OF PHENOLIC MONOESTERS OF HYDROXYMETHYL PHENOLS

(75) Inventors: Sanjay Jagdish DESAI, Ahmedabad (IN); Kalpesh Mahendrabhai PATEL, Ahmedabad (IN); Aakash Maheshkumar SHAH, Ahmedabad (IN); Ranjit Shardulbhai PADA, Ahmedabad (IN); Hitesh Manubhai MAKWANA, Ahmedabad (IN)

(73) Assignee: INTAS PHARMACEUTICALS LIMITED, Ahmedabad (IN)

(21) Appl. No.: 13/104,581

(22) Filed: May 10, 2011

(30) Foreign Application Priority Data
May 11, 2010 (IN) 1486/MUM/2010
Apr. 12, 2011 (IN) 1200/MUM/2011

(51) Int. Cl. C07C 213/00; C07C 215/54; C07C 39/12

(52) U.S. Cl. 560/252; 568/745; 564/336

(57) ABSTRACT

A process for the preparation of phenolic monoesters of 2-(3-diisopropylamino-1-phenylpropyl)-4-(hydroxymethyl)phenol by converting (±)6-halo-4-phenylchroman-2-one to (±)4-halo-2-(3-hydroxy-1-phenylpropyl)phenol. The two hydroxyl groups are protected and the protected compound is reacted with diisopropylamine to give (±)[3-(2-benzyloxy-5-halophenyl)-3-phenylpropyl]diisopropylamine. The halo substituent on the benzene ring is converted to corresponding benzyl alcohol and then the protection is removed to give racemic 5-HMT. Racemic 5-HMT is converted R enantiomer and then it is esterified.

### FIELD OF THE INVENTION

The present invention relates to a novel process for the preparation of phenolic monoesters of 2-(3-diisopropylamino-1-phenylpropyl)-4-(hydroxymethyl)phenol specifically fesoterodine or its fumarate salt which is a prodrug of tolterodine.

The present invention also provides metal salts of (+)-5-HMT, R-(+)-2-(3-diisopropylamino-1-phenylpropyl)-4-hydroxy methyl phenol an intermediate in the synthesis.

### BACKGROUND OF THE INVENTION

In man, normal urinary bladder contractions are mediated, (inter alia), through cholinergic muscarinic receptor stimulation. Muscarinic receptors not only mediate normal bladder contractions, but may also mediate the main part of the contractions in the overactive bladder resulting in symptoms such as urinary frequency, urgency and urge urinary incontinence.

U.S. Pat. No. 6,713,464 disclosed a variety of phenolic monoesters derivatives, processes for their preparation, pharmaceutical compositions and method of use thereof. These compounds are anti-muscarinic agents with superior pharmacokinetic properties compared to existing drugs such as oxybutynin and tolterodine and useful in the treatment of urinary incontinence, gastrointestinal hyperactivity (irritable bowel syndrome) and other smooth muscle contractile conditions. Among them, Fesoterodine, chemically 2-[(1R)-3-[bis(1-methylethyl)amino]-1-phenylpropyl]-4-hydroxymethylphenylisoburyrate is a new, potent and competitive muscarinic antagonist and useful in the potential treatment of urinary incontinence. It has the formula (I) depicted below.

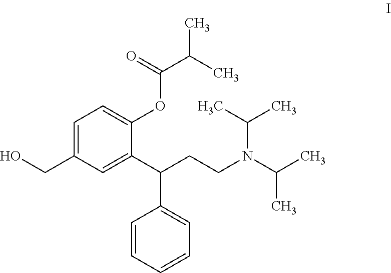

A synthetic approach for the production of the Active Metabolite and monoesters of the phenolic hydroxy group of the Active Metabolite such as Fesoterodine has been described in U.S. Pat. No. 6,713,464 which, is as follows:

Fesoterodine is prepared by reaction of (±)-6-bromo-4-phenylchroman-2-one with benzyl chloride in the presence of sodium iodide and anhydrous potassium carbonate in methanol and acetone to produce (±)-3-(2-benzyloxy-5-bromophenyl)-3-phenylpropionic acid methyl ester as a light yellow oil. This product is reduced with lithium aluminium hydride to produce (±)-3-(2-benzyloxy-5-bromophenyl)-3-phenylpropan-1-ol, which is then treated with p-toluenesulphonyl chloride in the presence of pyridine in dichloromethane to afford (±)-toluene-4-sulphonic acid 3-(2-benzyloxy-5-bromophenyl)-3-phenylpropyl ester. This product is then reacted with N,N-diisopropylamine in acetonitrile at reflux temperature for 97 hours to produce (±)-[3-(2-benzyloxy-5-bromophenyl)-3-phenylpropyl]-diisopropylamine as a brown and viscous syrup. This product is resolved to produce (R)-[3-(2-benzyloxy-5-bromophenyl)-3-phenylproopyl]diisopropylamine which is then subjected to Grignard reaction with ethyl bromide and magnesium to produce (±)-4-benzyloxy-3-(-3-diisopropylamino-phenylpropyl)-benzoic acid hydrochloride, followed by esterification with methanol in the presence of sulphuric acid to produce (R)-4-benzyloxy-3-(3-diisopropylamino-1-phenylpropyl)-benzoic acid methyl ester, this product is reduced with lithium aluminium hydride to produce (R)-[4-benzyloxy-3-(3-diisoprylamino-1-phenylpropyl)-phenyl]-methanol, which is then subjected to deprotection with Raney-Nickel to produce (R)-2-(3-diisopropylamino-1-phenylpropyl)-4-hydroxymethylphenol followed by condensation with isobutyryl chloride to give fesoterodine.

WO 94/11337 also describes a multi-stage process to synthesize the precursor to the Active Metabolite.

U.S. Pat. No. 6,809,214 discloses a process wherein compound of following formula is used for preparation of 3,3-diaryl propylamine derivative.

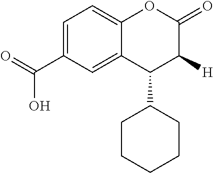

U.S. Pat. No. 6,858,650 discloses stable salts of 3,3-diphenylpropylamine derivative, the process of '650 utilizes R-(−)-3-(3-diisopropylamino-phenylpropyl)-4-hydroxy-benzoic acid methyl ester in the preparation of fesoterodine.

WO 2009/037569 discloses a process for preparation of fesoterodine. The process includes reaction of 4-phenylchromen of following formula:

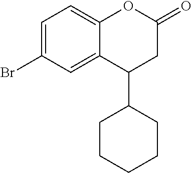

with benzyl chloride to give a compound of following formula which is further converted to fesoterodine (I) through multiple steps.

These previously described methods for producing the Active Metabolite require numerous steps that result in complex purification procedures, time-delay, and enhanced possibility of human error, thereby prohibiting optimal efficiency and cost-effectiveness.

Conversion of fesoterodine base into its fumarate salt is reported in U.S. Pat. No. 6,858,650, WO 2007/140986 and US 2010/0152483. The problem associated with prior art process is long time is required for isolation of crystals still this purity level can not be considered satisfactory as compared to pharmacopeias requirement for this product. To overcome one or more insufficiencies of prior art processes the present invention provides a process for preparation of crystalline fesoterodine fumarate which is easy to handle on industrial scale and provides a product with high purity complying with the pharmacopial limits.

### OBJECTS OF THE INVENTION

The main object of the invention to provide an improved process for the preparation of phenolic monoesters of 2-(3-diisopropylamino-1-phenylpropyl)-4-(hydroxymethyl)phenol, specifically fesoterodine or its fumarate salt

A further object of the present invention is to provide cost effective process for the preparation of phenolic monoesters of 2-(3-diisopropylamino-1-phenylpropyl)-4-(hydroxymethyl)phenol.

A further object of the present invention is to provide metal salts of (+)-5-HMT.

Another object of the present invention is to provide a process for the preparation of crystalline fesoterodine fumarate.

### SUMMARY OF THE INVENTION

The present invention relates to a novel process for the preparation of phenolic monoesters of 2-(3-diisopropylamino-1-phenylpropyl)-4-(hydroxymethyl)phenol by converting (±)-6-halo-4-phenylchroman-2-one to (±)-4-halo-2-(3-hydroxy-1-phenylpropyl)phenol. The two hydroxyl groups are protected and the protected compound is reacted with diisopropylamine to give (±)[3-(2-benzyloxy-5-halophenyl)-3-phenylpropyl]diisopropylamine. The halo substituent on the benzene ring is converted to corresponding benzyl alcohol and then the protection is removed to give racemic 5-HMT. Racemic 5-HMT is converted R enantiomer and then it is esterified.

In another aspect the present invention provides novel metal salts of formula (XII) and process for their preparation.

In yet another aspect the process of present invention provides preparation of compound of formula (VI) by reacting compound of formula (V) with diisopropylamine in water or in polar aprotic solvent and metal iodide or in absence of solvent.

In another aspect the present invention provides process to prepare compound of formula (VIII) by reduction of compound of formula (VII) in presence of borane containing reducing agents.

In a further aspect the present invention provides a process for crystallisation of compound of formula (IX) by using solvent selected from alkanols, ketones, esters, aromatic hydrocarbons, halogenated solvents, nitrile, water or mixture thereof.

In a further aspect the present invention provides improved method for isolation of intermediates of formula (VI) and (IX).

In a further aspect the present invention provides a process for preparation of crystalline fesoterodine fumarate.

### DETAILED DESCRIPTION

The following is the detailed description of preferred process according to the invention for preparing phenolic monoesters of 2-(3-diisopropylamino-1-phenylpropyl)-4-(hydroxymethyl)phenol.

According to one aspect of the invention there is provided a method for the preparation of phenolic monoesters of 2-(3-diisopropylamino-1-phenylpropyl)-4-(hydroxymethyl)phenol, specifically fesoterodine which comprises the following steps:

The starting material in the process of the present invention is (±)-6-halo-4-phenylchroman-2-one having formula (II). One method for preparing bromo compound is described in Example 1 of the WO 2009/037569, and involves the reaction of cinnamic acid and 4-bromophenol in the presence of sulphuric acid.

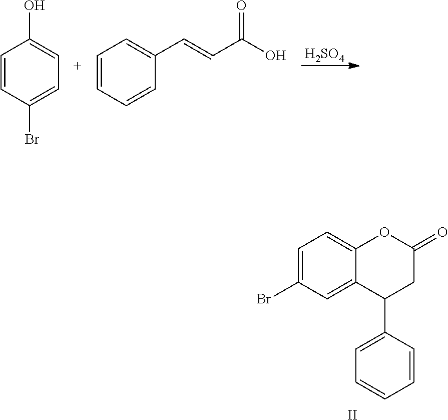

The first step in the process according to the present invention comprises reduction of (±) 6-halo-4-phenylchroman-2-one with reducing agent.

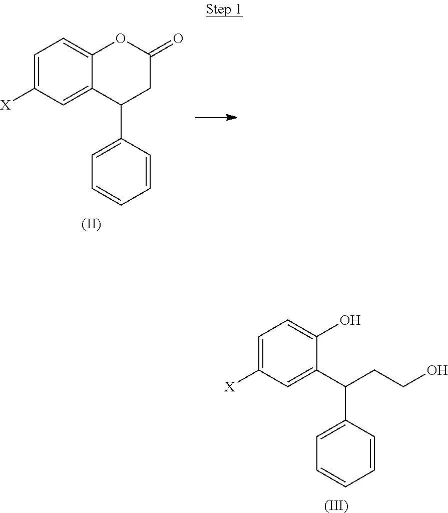

The reducing agent for step 1; can be selected from sodium borohydride, lithium borohydride or like. The reaction is carried out in presence of solvent. The solvent used for step 1, can be selected from cyclic ether such as tetrahydrofuran.

Although “X” in compound of formula (II) can be selected from any halo atom preferably 6-halo derivative i.e. (±)6-bromo-4-phenylchroman-2-one is used for the purpose of present invention.

The reaction can be carried out at higher temperature but preferably it is carried out at around ambient temperature. The reaction is carried out at 0-40° C. preferably at about 10-35° C.

The product of step 1 can be isolated by solvent extraction and pH adjustment. The solvents used for isolation of the product i.e. (±)4-bromo-2-(3-hydroxy-1-phenylpropyl)phenol (III) can be selected from any organic solvent preferably from a group comprising of ether like diisopropylether, aliphatic or aromatic hydrocarbon like hexane, cyclohexane, n-heptane, and mixture thereof.

The second step involves protection of the hydroxyl group of the phenol by reacting (±)4-bromo-2-(3-hydroxy-1-phenylpropyl)phenol with benzyl bromide or benzyl chloride in the presence of any base or acid scavenger and organic solvent to give (±)-3-(2-benzyloxy-5-bromophenyl)-3-phenylpropanol.

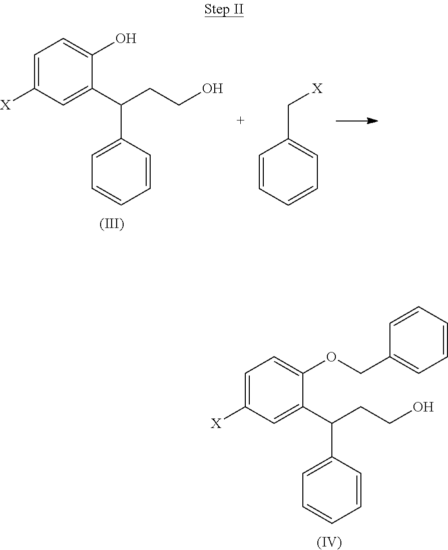

Preferably the base or the acid scavenger can be selected from any metal carbonate or hydroxide such as potassium carbonate, sodium carbonate, sodium hydroxide. The reaction can be carried out in any solvent but is preferably carried in presence of the solvent selected form the group comprising of ketone such as acetone, methyl isopropyl ketone, methyl-isobutyl ketone, methyl ethyl ketone or alkyl nitrile like acetonitrile. The product can be isolated from the reaction mixture by using organic solvent, such as ether, aliphatic or aromatic hydrocarbon or mixture thereof.

In third step, (±)-3-(2-benzyloxy-5-bromophenyl)-3-phenylpropanol is converted to (±) toluene-4-sulphonic acid or any other corresponding sulphonic ester 3-(2-benzyloxy-5-bromophenyl)-3-phenylpropyl ester by a process mentioned in U.S. Pat. No. 6,713,464 B1.

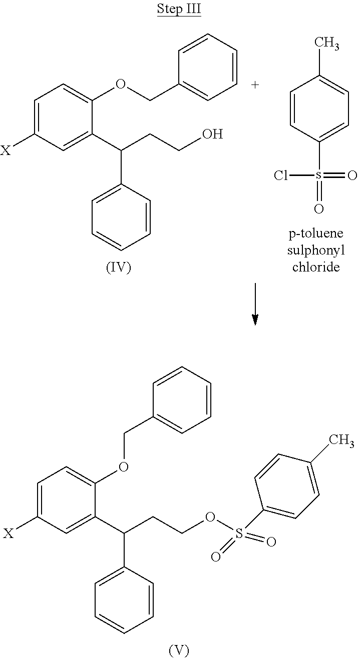

In fourth step, (±) toluene-4-sulphonic acid 3-(2-benzyloxy-5-bromophenyl)-3-phenylpropyl ester or any other corresponding sulphonic ester obtained in step III is reacted with diisopropylamine to give (±)-[3-(2-benzyloxy-5-bromophenyl)-3-phenylpropyl]-diisopropylamine (VI).

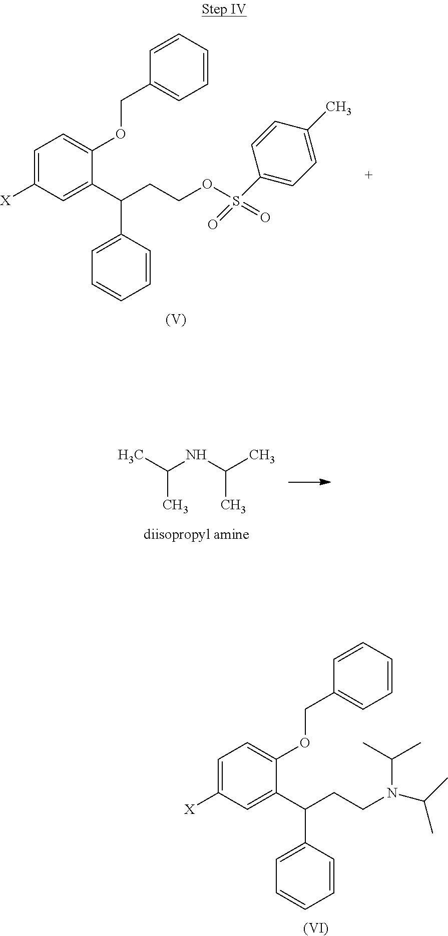

The reaction can be carried in the absence of solvent, or using water as solvent or in the presence of aprotic solvents and metal iodide. The aprotic solvent can be selected from N,N-diisopropylamine, N,N-dimethylformamide. The alkali metal used in the process can be selected from potassium iodide or sodium iodide. The reaction can be carried out at temperature in range of 80-150° C. preferably 90-110° C. The product is isolated by extraction with solvent and pH adjustment of the organic layer. The solvent used for extraction can be selected from any suitable organic solvent. In the process of present invention the pH of organic layer is adjusted by using orthophosphoric acid. The reported processes generally use sulphuric acid for pH adjustment (acid-base purification) this results into sulphate salt of compound of formula (VI). The sulphate salt is difficult to remove as it does not separate in aqueous layer rather forms an oily layer which is difficult to isolate and also entraps many impurities. When orthophosphoric acid is used the resultant salt does not isolate as oil rather goes in aqueous layer and can be isolated easily by further processing and is obtained with high purity.

The fifth step, of the process is to convert (±)-[3-(2-benzyloxy-5-bromophenyl)-3-phenylpropyl]-diisopropylamine to (±)-4-benzyloxy-3-(3-diisopropylamino-1-phenylpropyl)-benzoic acid hydrochloride by the process of U.S. Pat. No. 6,713,464B1.

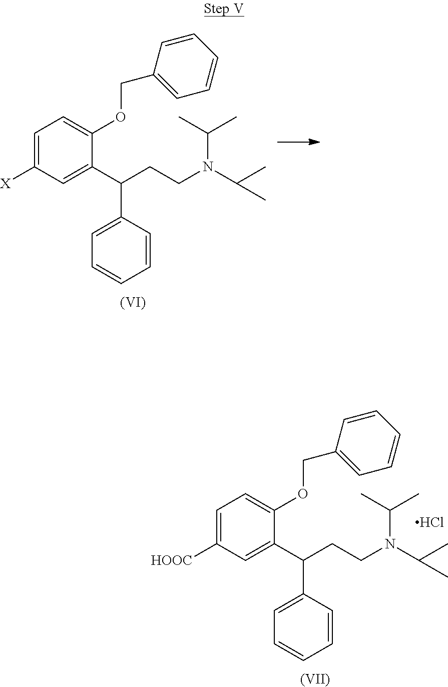

In step VI, (±)4-Benzyloxy-3-(3-diisopropylamino-1-phenylpropyl)-benzoic acid is converted by reduction to give (±)[4-Benzyloxy-3-(3-diisopropylamino-1-phenylpropyl)-Phenyl]-methanol.

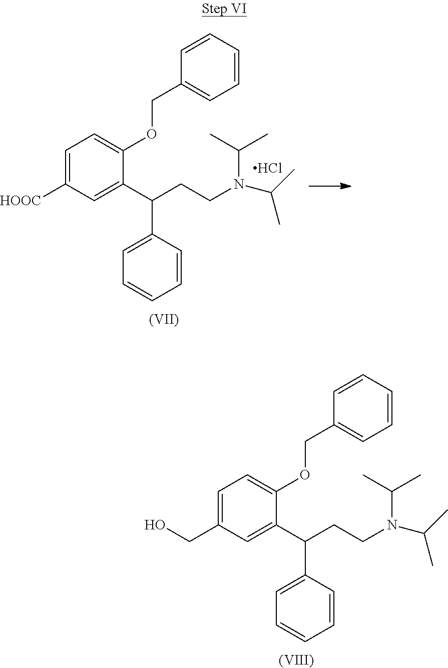

The reduction is performed by using borane containing reducing agents such BH₃.DMS (Boron Dimethyl sulphide complex), BH₃.THF (Boron THF).

The process of present invention provides direct reduction of acidic group of compound of formula VII, whereas the process of U.S. Pat. No. 6,713,464 discloses conversion of acid to ester by using methanol and sulphuric and then reduction of ester to alcohol. Thus the present invention avoids extra step and makes the overall process more feasible.

The reduction can be carried out in presence of solvent selected from group comprising aromatic hydrocarbon like toluene, cyclic ether like tetrahydro furan or mixture thereof. The product can be isolated by using organic solvents selected from group comprising of alkanols, esters, hydrocarbon, ketone, halogenated solvents, water or mixture thereof; preferably solvent for isolation is selected from alcohol like isopropyl alcohol.

In step VII, (±)[4-Benzyloxy-3-(3-diisopropylamino-1-phenylpropyl)-Phenyl]-methanol is debenzylated by known methods to give racemic 5-HMT.

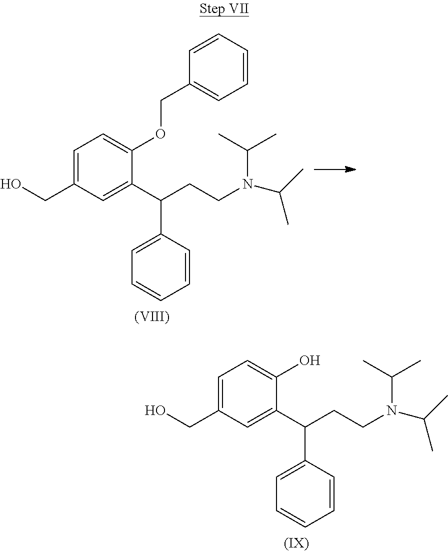

Compound of formula (IX) is difficult to be crystallised as solid by following the reported procedure and is generally obtained as liquid or oil. The process of present invention uses solvents selected from group comprising of alcohols, ethers, ester, aliphatic or aromatic hydrocarbon, halogenated solvents, ketones, nitriles or water. Use of these solvents enables crystallisation of compound of formula (IX) thus easing the isolation process.

Next step VIII relates to resolution of racemic compound of formula (IX). Resolution can be carried out by using a resolving agent like acetyl mandelic acid; in presence of any suitable organic solvent e.g. tetrahydrofuran. The process comprises reacting the racemic compound (IX) with acetyl mandelic acid to obtain salt of formula (X), treating the acetoxy mandelate salt of formula (X) with any suitable inorganic or organic base to liberate desired isomer i.e. (R)-(+)-2-(3-diisopropylamino-1phenylpropyl)-4-hydroxy methyl phenol of formula (XI). The compound of formula (XI) can be isolated from reaction mixture by distilling off the reaction solvent and treating the residue with a suitable solvent such as ketone like acetone, alkane like hexane, heptanes or mixture thereof

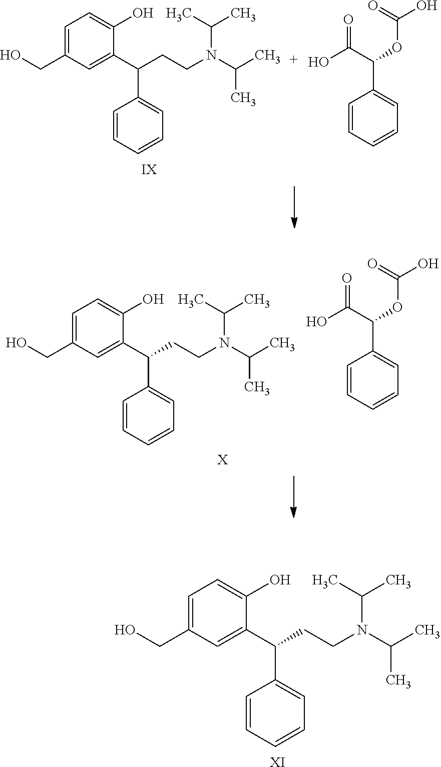

According to another aspect of the invention there is provided metal salts of compound of formula (XII) and can in general be represented by following structural formula.

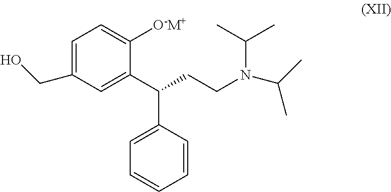

Wherein M⁺ represents any metal ion such as Na⁺, K⁺ or Li⁺

The metal salts of formula (XII) can be used in the preparation of fesoterodine. Alternatively the product of formula (IX) i.e. racemic 5-HMT can also be converted to its metal salt.

According to yet another aspect of the present invention there is provided a process for the preparation of the metal salts of formula (XII). For this the phenolic compound of formula (XI) is reacted with metal base such as sodium hydroxide, lithium hydroxide, potassium hydroxide or like. Solvent used for the reaction can be selected from any suitable organic solvent such as alcohol like methanol, isopropanol or ethanol; ketones like acetone, methyl ethyl ketone, methyl isopropyl ketone or methyl isobutyl ketone or mixture thereof. The reaction is carried out under heating preferably at a temperature in the range of 25-85° C. The metal salt of formula (XII) can be isolated from the reaction mixture by distilling off the reaction solvent followed by addition of solvent to the residue. The product of formula (XII) can be isolated from residue by adding solvent selected from aromatic hydrocarbon like toluene, halogenated solvent like dichloromethane, ether like tetrahydrofuran, diisopropyl ether, petroleum ether, alkane like cyclohexane. The solid thus obtained can be separated by filtration or similar technique and dried.

(+)-5-HMT or its metal salt is esterified to give phenolic monoesters of 2-(3-diisopropylamino-1-phenylpropyl)-4-(hydroxymethyl)phenol. According to the present invention the esterification can be done by the following methods:

1) Esterification of (+)-5-HMT in an organic solvent and in the absence of base
2) Esterification of (+)-5-HMT in an organic acid like isobutyric acid in the presence of coupling reagents like N,N-dicyclohexylcarbodiimide (DCC) or 1-hydroxybenzotriazole in an organic solvent.
3) Esterification of metal salts of (+)-5-HMT in presence of an organic solvent

The final step of the process is conversion of phenolic monoesters of 2-(3-diisopropylamino-1-phenylpropyl)-4-(hydroxymethyl)phenol, specifically fesoterodine to its fumarate salt.

According to the present invention, fesoterodine fumarate is prepared by reacting fesoterodine base with fumaric acid in presence of 2-butanone and using anti solvent selected from ether, alkane, aromatic hydrocarbons or mixtures thereof.

The fesoterodine fumarate can be further recrystallised using the same solvent system used for the preparation of salt.

The characteristic features of product obtained as per the above method matches with product obtained as per e.g. 6 of U.S. Pat. No. 6,858,650 and with Form I as disclosed in US 2010/0152483. For reference the comparative data is provided in tables given below.

TABLE 1
2 theta values of product obtained as per U.S. Pat. No. 6,858,650, process of
present disclosure and of US2010/0152483

| Following | | Following | | Process of present | |
|---|---|---|---|---|---|
| U.S. Pat. No. 6,858,650 B1 | | US2010/152483 A1 | | disclosure | |
| 2 Theta | | 2 Theta | | 2 Theta | |
| Value | Intensity | Value | Intensity | Value | Intensity |
| 4.4 | 3.7 | 4.5 | 1.95 | 4.4 | 2.7 |
| 8.79 | 14.2 | 8.94 | 8.22 | 8.78 | 9.9 |
| 10.37 | 87.8 | 10.55 | 23.71 | 10.37 | 91.3 |
| 11.52 | 47.6 | 11.63 | 13.72 | 11.52 | 52.2 |
| 11.8 | 58.2 | 11.93 | 19.95 | 11.82 | 50.2 |
| 12.85 | 37 | | | 12.83 | 43.2 |
| 13.2 | 20.7 | 13.01 | 17.71 | 13.19 | 15.9 |
| 14.86 | 9.8 | 13.31 | 17.9 | 14.89 | 8.5 |
| 15.1 | 7.7 | 15.09 | 2.95 | 15.09 | 8.3 |
| 15.29 | 2.2 | | | | |
| 15.89 | 11 | | | 15.9 | 10.5 |
| 16.21 | 18.8 | | | 16.2 | 21.8 |
| 16.54 | 19.2 | 16.42 | 10.37 | 16.55 | 25.1 |
| 17.64 | 67.9 | 17.74 | 61.68 | 17.61 | 55.8 |
| 17.97 | 33.4 | 18.1 | 23.67 | 17.95 | 21.3 |
| 18.81 | 56.4 | | | | |
| 18.96 | 85.2 | | | 18.96 | 100 |
| 19.32 | 27.8 | 19.14 | 27.82 | 19.31 | 31.6 |
| | | 19.59 | 9.37 | | |
| 19.98 | 36.8 | 20.06 | 24.45 | 19.99 | 38.4 |
| | | 20.16 | 30.05 | | |
| 20.79 | 56.4 | 20.98 | 16.27 | 20.79 | 74.1 |
| 22.07 | 100 | 22.2 | 100 | 22.06 | 82.5 |
| | | | | 22.82 | 16.8 |
| 23.05 | 36.1 | | | 23.06 | 37.7 |
| 23.33 | 6.7 | 23.25 | 15.13 | 23.33 | 7.1 |
| 23.75 | 9 | | | 23.82 | 8.1 |
| 24.09 | 13.1 | | | 24.05 | 17 |
| | | 24.16 | 8.76 | 24.1 | 17.7 |
| 24.92 | 21.2 | 25.07 | 7.36 | 24.93 | 21.3 |
| | | | | 25.3 | 5.9 |
| 25.53 | 9.3 | | | 25.55 | 10.3 |
| 25.89 | 32.4 | | | 25.86 | 37 |
| | | 26.12 | 23.08 | 26.17 | 3.3 |
| 27.01 | 7 | | | 27.02 | 8 |
| 27.46 | 5.4 | | | | |
| 27.74 | 10.1 | | | 27.73 | 14.6 |
| | | | | 28.06 | 14.2 |
| 28.2 | 20.4 | 28.34 | 21.45 | 28.2 | 22.2 |
| 29.17 | 3.3 | | | 29.14 | 3.2 |
| | | 29.44 | 3.04 | | |
| 30.46 | 3.7 | | | 30.46 | 4.1 |
| 30.78 | 5.5 | 31.07 | 2.27 | 30.77 | 6.5 |
| | | | | 31.22 | 2 |
| 31.64 | 6.2 | 31.96 | 2.21 | 31.65 | 7.4 |
| 32.51 | 12.5 | 32.66 | 12.4 | 32.5 | 113.9 |
| 32.81 | 3.9 | | | 33.3 | 1.5 |
| 34.98 | 2.1 | | | | |
| 35.73 | 2.5 | 35.83 | 2.37 | 35.75 | 2.7 |
| 36.28 | 5.2 | 36.46 | 2 | 36.26 | 6 |
| 36.93 | 3.9 | 37.01 | 2.72 | 36.92 | 3.6 |
| | | | | 37.35 | 2.1 |
| 38.44 | 1.5 | | | | |
| 38.65 | 2.3 | | | 38.68 | 2.2 |
| | | | | 39.02 | 3.4 |
| 39.23 | 3.5 | | | 39.2 | 3.9 |
| 40.07 | 2.1 | | | 40.12 | 1.9 |
| 40.59 | 2.8 | | | 40.59 | 3.3 |
| 41.47 | 1.9 | | | 41.46 | 2.1 |
| 42.34 | 1.8 | | | 42.29 | 2 |
| 44.83 | 1.7 | | | | |
| 45.05 | 2.3 | | | 45.05 | 2.4 |
| 45.3 | 2 | | | 45.33 | 2.2 |

TABLE 2
DSC values of product obtained as per U.S. Pat. No. 6,858,650, process of
present disclosure and of US2010/0152483

| Sr. No | Reference | DSC Value | Solvents used |
|---|---|---|---|
| 1 | Following U.S. Pat. No. | 105.7 to | 2-butanone & |
| | 6,858,650 | 111.55 | cyclohexane |
| 2 | Following | 105.95 to | As disclosed therein |
| | US2010/0152483 A1 | 109 | |
| 3 | Process of present | 105.22 to | 2-butanone & |
| | disclosure | 109.73.17 | Diisopropyl ether |

TABLE 3
IR frequencies of product obtained as per U.S. Pat. No. 6,858,650, process of
present disclosure and of US2010/0152483

| Following | Following | Process of present |
|---|---|---|
| U.S. Pat. No. 6,858,650 B1 | US2010/0152483 | disclosure |
| Wave length (1/cm) | Wave length (1/cm) | Wave length (1/cm) |
| 3481 | 3473 | 3473 |
| 3412 | 1757 | |
| 3373 | 1700 | |
| 3348 | 1558 | 3331 |
| | 1234 | |
| 1757 | 1213 | 1755 |
| 1701 | 1176 | 1691 |
| 1544 | 1156 | 1680 |
| 1629 | 1127 | 1581 |
| 1232 | 1097 | 1226 |
| 1213 | 1018 | 1215 |
| 1176 | 982 | 1176 |
| 1157 | 907 | 1128 |
| 1126 | 794 | 1095 |
| 1097 | 703 | 1020 |
| 1064 | | 983 |
| 1037 | | 910 |
| 1020 | | 794 |
| 981 | | 746 |
| 939 | | 702 |
| 908 | | |
| 792 | | |
| 748 | | |
| 702 | | |

The following examples are illustrative of the invention but not limitative of the scope thereof.

### EXAMPLES

### Example 1

### Preparation of (±)-4-bromo-2-(3-hydroxy-1-phenylpropyl)phenol (III)

6-bromo-4-phenylchroman-2-one (900 g) and tetrahydrofuran (10.8 litres) were taken into a reaction flask. All the contents were cooled to 0-5° C. This was followed by addition of sodium borohydride (14200 g), after completion of addition the temperature of reaction mixture was raised to 25-35° C. The reaction mass was stirred at the same till completion of reaction. Reaction was monitored by TLC. After completion of reaction, the reaction mixture was cooled to 0-5° C., to this water (1.8 litre) was added and the solvent was distilled off. Again the reaction mass was cooled to 5-15° C. followed by addition of water (5.4 litre); the pH of resultant mixture was adjusted to 1-2 by using hydrochloric acid. The reaction mixture was stirred for 30 minutes at 25 to 30° C., followed by addition of ethyl acetate 5.4 lit, the layers were separated, and organic layer was washed with sodium chloride solution, followed by distillation of solvent. To the residue thus obtained 0.5 volume of toluene was added and distilled off, followed by second addition of toluene (4.5 lit) to the residue, the reaction mixture thus obtained was heated to 50-55° C. and then cooled to 25-30° C. and stirred at the same for about 40 minutes. The reaction mixture was further cooled to 0-10° C. and maintained at the same for 60-65 minutes; the solid thus separated was filtered and dried to obtain 8750 g. of title compound.

Purity 98.2%

Mass-LC-MS 306.83 (M-H);

NMR (400 MHz, DMSO-d₆) δ 2.15 (q, 2, OCH2CH2), 3.35 (m, 2, OCH2CH2), 4.45 (t, 1, CHCH2), 4.52 (s, 1, OH), 6.76 (d, 1, H—Ar), 7.13-7.39 (m, 7, H—Ar), 9.73 (s, 1, OH, Phenolic)

### Example 2

### Preparation of (±)-3-(2-benzyloxy-5-bromophenyl)-3-phenyl propanol (IV)

8700 g of 4-bromo-2-(3-hydroxy-1-phenylpropyl)phenol, 52.2 litre of acetone and 5860 g of potassium carbonate were taken in a reaction flask and the contents were stirred for 30-35 minutes at 20-25° C. This was followed by addition 5090 g of benzyl bromide, the reaction mixture was heated to 50° C. and was stirred at 55-60° C. till completion of the reaction. After completion of reaction the organic solvent was distilled off and the reaction mass was cooled to 30° C. The residue was extracted from methylene dichloride, and the solvent from organic layer was distilled off. The product was isolated by adding 1.2 vol. of diisopropyl ether and

5.0 volume of hexane, the product thus separated was filtered and dried to obtain 10050 g of the title compound.

Purity 97.16%

### Example 3

### Preparation of (±) toluene-4-sulphonic acid 3-(2-benzyloxy-5-bromophenyl)-3-phenyl propyl ester (V)

10500 g of 3-(2-benzyloxy-5-bromophenyl)-3-phenyl propanol, 26.5 litre methylene dichloride were charged in a reaction flask and the contents were stirred at the 25-30° C. for 30 minutes. To this 7500 g of pyridine was added and the reaction mixture was cooled to 5-10° C. To this solution of 5500 g of p-toluene sulfonyl chloride in 26.5 lit of methylene dichloride was added. The reaction mass was stirred at the 25-30° C. till completion of reaction. After completion of reaction the reaction mixture was cooled to 10° C. and 10 vol. of hydrochloric acid was added to this. The layers were separated, and the solvent from the organic layer was distilled off. The title product was obtained as oil.

Purity 81.18%

### Example 4

### Preparation of (±)-[3-(2-benzyloxy-5-bromophenyl)-3-phenylpropyl]-diisopropylamine (VI)

Toluene-4-sulphonic acid-3-(2-benzyloxy-5-bromophenyl)-3-phenylpropyl ester 13500 g, 67.5 litre of diisopropylamine, 40.5 lit of water were taken in a reaction flask. The reaction mass was heated to 75-78° C. After completion of reaction the reaction mass was cooled to 25-30° C. and the reaction mass was extracted from diisopropylether, layers separated, and the pH of organic layer was adjusted to 1-2 by using orthophosphoric acid. The aqueous layer was separated from the reaction mass and treated with base (sodium hydroxide solution) and then the reaction mass was extracted with diisopropyl ether, layers were separated, solvent was distilled from the organic layer to give 8200 g of the title product is obtained as oily mass.

Purity 88.76%

### Example 5

### Preparation of (±)-4-benzyloxy-3-(3-diisopropylamino-1-phenylpropyl)-benzoic acid hydrochloride (VII)

In a reaction flask 700 ml of tetrahydrofuran, 93.75 g of magnesium turning and iodine were charged and to this mixture 26.80 ml of ethyl bromide was added at 25-30° C. The reaction mixture was heated to 50-60° C. and stirred at the same for 60 minutes. To this reaction mixture solution of [3-(2-benzyloxy-5-bromophenyl)-3-phenylpropyl]-diisopropylamine (350 g) in 153.2 ml of ethyl bromide and 1750 ml of tetrahydrofuran was added, the temperature was maintained at about 50-60° C. and reaction mixture was stirred at the same for 90-95 minutes. The reaction mixture was cooled to −70° C. to −60° C. and at this temperature 700 g of dry ice was added. After completion of reaction the temperature was increased to −50° C. and 2800 ml. of 20% ammonium, chloride was added to the reaction mixture. Layers were separated and pH of organic layer was adjusted to 1.0; the reaction mixture was stirred at the same pH for 12-13 hours at 25-30° C., filtered and dried to obtain 248 g, of title compound.

Purity 91.88%

### Example 6

### Preparation of (±)-[4-benzyloxy-3-(3-diisopropylamino-1-phenylpropyl)-phenyl]-methanol (VIII)

375 g of (±)-4-benzyloxy-3-(3-diisopropylamino-1-phenyl propyl)-benzoic acid hydrochloride, tetrahydrofuran 2625 ml, were taken in a reaction flask To this BH₃.DMS (118.08 g) was added at 25-30° C., the reaction mixture was heated to 45-50° C. and stirred at the same till completion of reaction. Once the reaction is complete the reaction mixture was cooled to 10-15° C. followed by addition of methanol 375 ml and 7500 ml sulphuric acid, the reaction mixture was stirred for 30 minutes followed by addition of 3750 ml of cyclohexane, reaction mixture stirred for 30 minutes and layers were separated. The aqueous layer was extracted from methylene dichloride and the organic thus separated was cooled to 0-5° C., the solid isolated at this temperature was separated by filtration and dried to obtain 175 g of title compound.

Purity 96.39%

### Example 7

### Preparation of (±)-2-(3-diisopropylamino-1-phenyl propyl)-4-hydroxy methyl phenol (IX)

55 g of (±)-[4-benzyloxy-3-(3-diisopropylamino-1-phenylpropyl)-phenyl]-methanol and 550 ml of methanol were taken in a flask and charged in an autoclave with 27.5 g of raney nickel. Reaction mixture was stirred at 30-35° C. till completion of reaction. After completion, the reaction mixture was filtered to remove catalyst, and the filtrate was distilled under vacuum at 40-45° C. To the residue acetone 82.5 ml was added, and stirred at 40-45° C. to get clear solution, to this n-heptane 440 ml was added and the reaction mixture was stirred for 6 hours at 25-30° C. and then at 0-5° C. for 2 hours, the reaction mixture was filtered and the solid thus obtained was dried to get 34.8 g of title compound.

Purity 98.29%

### Example 8

### Preparation of R-(+)-2-(3-diisopropylamino-1-phenylpropyl)-4-hydroxymethyl phenol acetoxy mandelic acid salt (X)

In a reaction flask 30 g of (±)-2-(3-diisopropylamino-1-phenylpropyl)-4-hydroxymethylphenol and 210 ml of tetrahydrofuran was taken and the resultant mixture was heated to 50-55° C. To this solution of R-(−) acetoxy mandelic acid in 60 ml tetrahydrofuran, was added. The resultant mixture was stirred for 6 hours at 25-30° C. and then was cooled to 10-15° C. and stirred for 2-3 hours at the same temperature. The solid thus obtained was separated by filtration and the product is dried to get 19.8 g of compound.

Purity 99.5%

### Example 9

### Preparation of R-(+)-2-(3-diisopropylamino-1-phenylpropyl)-4-hydroxy methyl phenol (XI)

19 g of R-(+)-2-(3-diisopropylamino-1-phenylpropyl)-4-hydroxymethyl phenol acetoxy mandelic acid salt, 190 ml toluene, 19 g of potassium carbonate and 190 ml of water were taken in a reaction flask, the contents were heated to 50-55° C. for 60-90 minutes, layers separated and the aqueous layer was washed with toluene and the organic layer was washed with water, toluene was distilled from collected organic layer 38 ml of acetone was added to the residue and the reaction mixture was heated 40-45° C. for 45 minutes followed by addition of n-heptane 342 ml, reaction mixture was cooled to 5-10° C. and the product thus separated was filtered and dried to obtain 9.0 g of title compound.

Purity 99.80%

### Example 10

### Preparation of metal salt of R-(+)-2-(3-diisopropylamino-1-phenylpropyl)-4-hydroxy methyl phenol provide details (XII)

R-(+)-2-(3-diisopropylamino-1-phenylpropyl)-4-hydroxy methyl phenol (XI) 5.0 gm, 50 ml of methanol and 0.35 gm of lithium hydroxide were charged in a reaction flask. The reaction mixture was heated at 50-60° C. for 10-15 hours. After completion of reaction the solvent is distilled off and to the residue 50 ml of toluene was added. The reaction mixture was stirred for 60 minutes at 25-30 C. The solid thus separated was filtered and dried to give 4.5 g of title compound.

Mass LC-MS 340.57 (M-Li);

NMR (400 MHz, DMSO-d6) δ 0.83 (m, 12, CH(CH3)2), 2.02 (m, 2, NCH2CH2), 2.30 (m, 2, NCH2CH2), 2.91 (m, 2, CH(CH3)2), 4.36 (m, 2, OCH2), 4.90 (m, 1, CHCH2), 6.97-7.33 (m, 8, H—Ar)

### Example 11

### Preparation of Fesoterodine (I)

28 gm of R (+)5-HMT (XI) and 280 ml of methylene dichloride were taken in a reaction flask and stirred 25-30° C. Reaction mixture was cooled to −20 to −25° C. Solution of isobutyryl chloride (9.48 g) in 140 ml of methylene dichloride was added to the reaction mixture while maintaining the temperature at −20° C. to −25° C. Reaction mixture stirred at −20° C. to −25° C. till completion of reaction. 280 ml of distilled water was added to the reaction mixture at −15° C. to −20° C., and stirred at 30 min at 10-15° C. for 30 minute, layers were separated. The solvent from organic layer was distilled off to obtain 31.0 g of title compound.

### Example 12

### Preparation of Fesoterodine Fumarate

30.8 gm of fesoterodine base, 184.8 ml of methyl ethyl ketone and 8.6 g of fumaric acid were charged in a reaction flask. Reaction temperature was increased to 35±5° C. and stirred at the same till a clear solution is obtained, after that 61.6 ml of cyclohexane was added. Reaction mixture was cooled to 25-30° C. and was stirred at the same for 5-6 hours. Reaction mixture was cooled to 0-5° C. and stirred at the same for stir for 24 hours. The solid thus separated was filtered and directly taken for purification.

### Example 13

### Recrystallisation of Fesoterodine Fumarate Salt

Crude fesoterodine fumarate salt as obtained form example 12 and 184.8 ml of methyl ethyl ketone were charged in a reaction flask and the temperature was increased to 45±5° C., stirred clear solution was obtained. After this 61.6 ml of cyclohexane was added to the reaction mixture. After complete addition of cyclohexane reaction mixture was cooled to 25-30° C., stirred for 5-6 hours at 25-30° C. reaction mixture was cooled to 0-5° C., and stirred at the same for 2-3 hrs. The solid thus separated was filtered and dried to obtain 30.0 gm of title compound.

Purity of 99.51%.

### Example 14

### Preparation of Crystalline Fesoterodine Fumarate

To a solution of (R)-(+)-2-(3-diisopropylamino-1-phenylpropylamino-1-phenylpropyl)-4-hydroxymethyl phenyl isobutyrate ester (fesoterodine base) 10 g, in 60 ml 2-butanone, fumaric acid 2.82 g was added. Followed by heating at 30-40° C. to form clear solution; 30 ml of diisopropylether was added at the same temperature over a period of 30 min. The reaction mass was cooled to 20-25° C. and stirred at the same for 5-6 hours. The reaction mass was further cooled to 0-5° C. and stirred at the same for 5-6 hours. The separated solid was collected by filtration and washed with 20 ml mixture of diisopropylether: 2-butanone (9:1), and then dried for 24 hrs under vacuum at below 35° C. to obtain 8.6 g of title compound.

Purity 99.6%

### Example 15

### Recrystallization of Crystalline Fesoterodine Fumarate

8.6 g of crystalline fesoterodine fumarate as obtained from example 1, is dissolved in 51.6 ml 2-butanone. The temperature of reaction mass is raised to 30-40° C., after complete dissolution, diisopropylether 25.8 ml is added to the reaction mass at same temperature. After addition of diisopropylether reaction temperature is cooled to 20-25° C. and stirred at the same for 5-6 hours. The reaction mass was further cooled to 0-5° C. and stirred at the same for 5-6 hours. The separated solid was filtered, washed with 17.2 ml mixture of diisopropylether: 2-butanone (9:1), and then dried for 24 hrs under vacuum at below 35° C. to obtain 7.3 g of pure fesoterodine fumarate.

### REFERENCES EXAMPLES

### Reference Examples are Repetition of Process Disclosed in Example 6 of U.S. Pat. No. '650

### Reference Example-1

### Preparation of Fesoterodine Base (I)

R)-(+)-2-(3-diisopropylamino-1-phenylpropylamino-1-phenylpropyl)-4-hydroxy methyl phenyl isobutyrate ester (fesoterodine base) 10 g and 21.5 ml of 2-butanone were taken in a round bottom flask, followed by addition 2.82 g of fumaric acid. The temperature of reaction mixture was raised to 60-65° C. After complete dissolution of fumaric acid cyclohexane 6.0 ml was added over a period of 30 min. at the same temperature while stirring. After addition of cyclohexane the temperature of reaction mixture was cooled to 25-30° C. followed by stirring for 18 hrs while maintaining the temperature. The reaction mixture was further cooled to 0-5° C. and left at the same for 18 hrs while stirring. The colorless crystals precipitate out and were separated by filtration. The product was washed with cyclohexane: 2-butanone (9:1) 20 ml, and then dried for 24 hrs under vacuum at below 35° C. to obtain 8.3 g of title compound.

### Reference Example-2

### Recrystallization of Crystalline Fesoterodine Fumarate

Fesoterodine fumarate (8.3 g) as obtained from reference example 1 was dissolved in 21.5 ml of 2-butanone while maintaining the temperature at 70-75° C. After obtainment of clear solution the temperature was cooled to 60-65° C., followed by addition of 6.0 ml cyclohexane over a period of 30 min while stirring. The temperature of reaction mixture was cooled to 25-30° C. and maintained at the same for 18 hrs under stirring. The reaction mixture was further cooled to 0-5° C. and left at the same for 18 hrs while stirring. The reaction mixture was filtered to separate the colorless crystals and washed with 16.2 ml mixture of cyclohexane: 2-butanone (9:1), and then dried for 24 hrs under vacuum at below 35° C. to obtain 7.3 g of pure fesoterodine fumarate.

Melting Point 103-108° C.

We claim:

1. A process for preparation of fesoterodine of formula (I) or its pharmaceutically acceptable salt comprising: 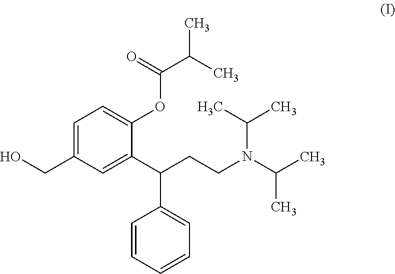
a) reducing compound of formula (II); 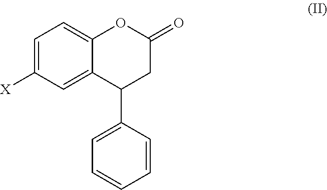
to give compound of formula (III), wherein ‘X’ represents a halogen atom 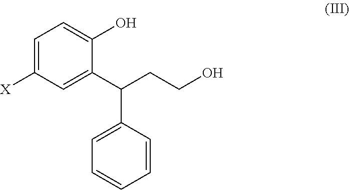
b) converting compound of formula (III) to (+)-2-(3-diisopropylamino-1-phenylpropyl)-4-hydroxy methyl phenol of formula (XI); 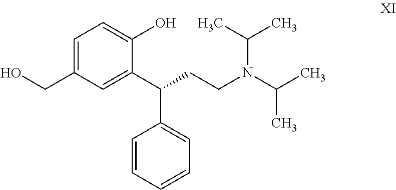
c) optionally converting compound of formula (XI) into its metal salt (XII), and then to fesoterodine (I) or its pharmaceutically acceptable salt.

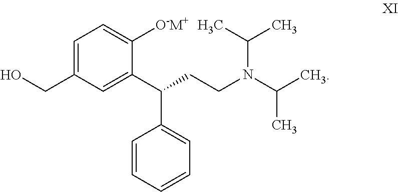

2. A process as claimed in claim 1, wherein the reaction in step a), is carried out in presence of reducing agent selected from sodium borohydride, lithium aluminium hydride or lithium borohydride.

3. A compound of formula (III): 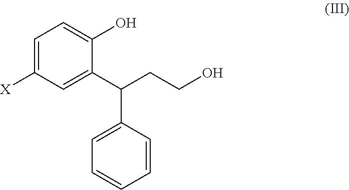
wherein X represents halogen atom.

4. A compound of formula

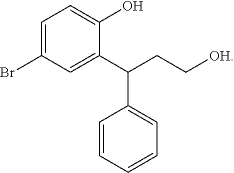

5. A process as claimed in claim 1; wherein conversion step b), comprises steps of:
1) reacting (±)-4-halo-2-(3-hydroxy-1-phenylpropyl)phenol of formula (III) with benzyl halide to give compound of formula (IV); 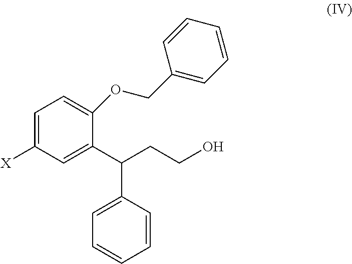
2) reacting compound of formula (IV) with sulphonic acid derivative to give compound of formula (V); 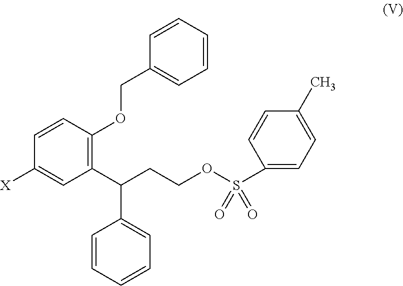
3) reacting compound formula (V), with diisopropylamine in absence of solvent or in presence of water or aprotic solvent and metal iodide to obtain compound of formula (VI); 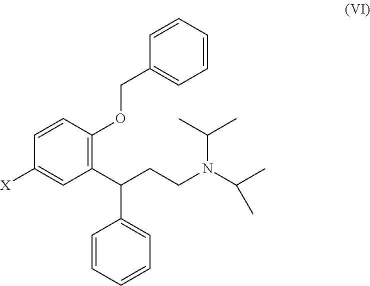
4) reacting compound of formula (VI) with ethyl bromide and magnesium to give (±)-4-benzyloxy-3-(3-diisopropylamino-1-phenyl propyl)-benzoic acid hydrochloride of formula (VII); 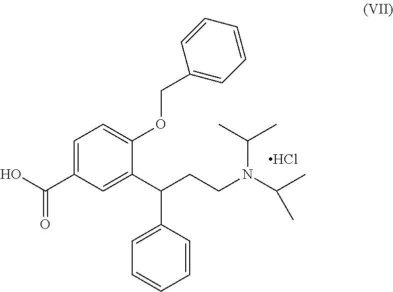
5) reducing compound of formula (VII) in presence of boron containing reducing agent to give compound of formula (VIII) 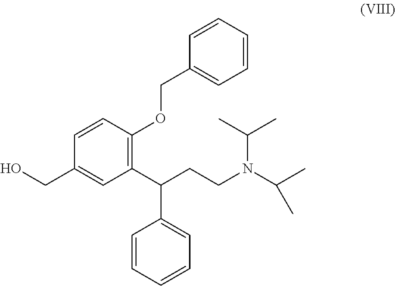
6) deprotecting compound of formula (VIII) to give (±)-2-(3-diisopropyl amino-1-phenylpropyl)-4-hydroxy methyl phenol of formula (IX) 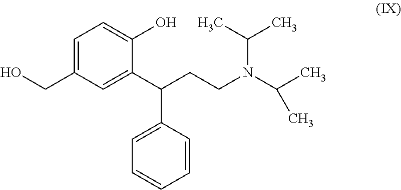
7) resolving the compound of formula (IX) obtained in step (f) with a suitable optically active acid to give (R) enantiomer of formula (XI);
8) Optionally reacting compound of formula (XI) with metal base to give metal salts of formula (XII);
9) Condensing the compound of formula (XI) or (XII) with isobutyryl chloride to give fesoterodine and optionally converting the fesoterodine into a pharmaceutically acceptable salt.

6. A process for preparation of fesoterodine (I) wherein compound of formula (IV) is prepared by reacting compound of formula (III) with benzyl halide.

7. A process to prepare fesoterodine wherein, compound of formula (VI) is prepared by reacting compound of formula (V) with diisopropylamine in water or in polar aprotic solvent and metal iodide or in absence of solvent.

8. A process to prepare fesoterodine wherein compound of formula (VIII) is prepared by reduction of compound of formula (VII) in presence of borane containing reducing agents.

9. A process for preparation of fesoterodine wherein compound of formula (VI) is isolated by using orthophosphoric acid.

10. A process for preparation of fesoterodine wherein compound of formula (IX) is crystallised by using solvent selected from alkanols, ketones, esters, aromatic hydrocarbons, halogenated solvents, nitrile, water or mixture thereof.

11. Metal salts of R-(+)-2-(3-diisopropylamino-1-phenylpropyl)-4-hydroxy methyl phenol of formula (XII)

12. A process to prepare metal salt of formula (XII) comprising reacting compound of formula (XI) with metal base.

13. A process as claimed in claim 12, wherein the metal base is selected from a group comprising of sodium hydroxide, potassium hydroxide or lithium hydroxide.

14. A process for the preparation of crystalline fesoterodine fumarate comprising, reacting fesoterodine base with fumaric acid in 2-butanone and anti solvent selected form ether, alkane, aromatic hydrocarbon or mixture thereof.

15. A process for recrystallisation of fesoterodine fumarate comprising, making solution of fesoterodine fumarate in 2-butanone and adding anti solvent selected form ether, alkane, aromatic hydrocarbon or mixture thereof.
